(19) United States
(12) Patent Application Publication
(10) Pub. No.: US 2025/0341483 A1
(43) Pub. Date: Nov. 6, 2025

(54) CATION EXCHANGE CAPACITY ESTIMATION SYSTEM AND METHOD

(71) Applicant: CGG SERVICES SAS, Massy Cedex (FR)

(72) Inventors: Stefan CALVERT, Crawley (GB); Sean HUMPHREYS, Llandudno (GB); Gareth ROGERS, Llandudno (GB)

(21) Appl. No.: 18/854,098

(22) PCT Filed: Apr. 12, 2022
(86) PCT No.: PCT/IB2022/000225

§ 371 Date: Oct. 4, 2024

(51) Int. Cl. G01N 23/2251; G01N 33/24

(52) U.S. Cl. CPC G01N 23/2251; G01N 33/24; G01N 2223/616

(57) ABSTRACT

A method for calculating a cation exchange capacity, CEC, associated with a mineral sample includes receiving a material sample, altering the material sample to become a mineral sample that fits into an automated mineralogy and petrography system, analyzing the mineral sample with the automated mineralogy and petrography system to generate a mineral map, selecting only clays from the mineral map, calculating a total surface area of the clays in the mineral map, and calculating the CEC value of the material sample based on the total surface area of the clays of the mineral map and for all minerals in the mineral map.

### BACKGROUND

### Technical Field

Embodiments of the subject matter disclosed herein generally relate to a system and method for estimating/calculating a cation exchange capacity (CEC) associated with a material sample, and more particularly, to using a non-destructive method for analyzing the material sample and determining, in a consistent way, the CEC value of the material sample.

### Discussion of the Background

Lateral (also known as “horizontal”) wells have proven particularly useful in the hydraulic fracking process employed in extracting gas and oil from shale reservoirs. These reservoirs tend to be inaccessible to traditional vertical drilling due to the impermeability of the shale formations. In order to extract gas and oil, a compound of water and chemicals, also known as mud, is pumped into lateral wells and into the surrounding formations. The force of these injections fractures the rocks in the formations, creating openings through which the petroleum flows from the formation into the well. The same method can be used in any type of well, e.g., vertical.

Geological and engineering parameters are used in well performance analysis to assess hydrocarbon productivity. Existing approaches to well performance analysis tend to rely on drilling and completion parameters and on geological information from well logs and rock samples (such as core plugs and cuttings). One such parameter is the electrical potential of the clay existing in the underground formation. This parameter is known as the cation exchange capacity (CEC). The CEC of clay minerals is a measure of their ability to adsorb cations from a solution. The CEC has been defined as the quantity of cations that are available at a given pH for exchange with other cations and is usually expressed in milliequivalents/100 g of dry clay. The adsorbed cation replaces or exchanges the original negative layer charge balancing cation in solution. This ability of colloidal particles, such as clay minerals, to retain and exchange positively charged ions is vital to the oil exploration as it has a controlling influence on the mobility of positively charged chemical species both in soils and geochemical cycling of cations in general.

CEC is normally associated with clay minerals with interlayer exchangeable cations such as smectites. The CEC process is a reversible process. It is also stoichiometric and diffusion controlled. Currently, the CEC is calculated via several different methods. For lithified rock samples, the CEC analysis encompasses (1) the crushing of solid samples for whole rock and (2) clay fraction X-ray diffraction (XRD) and laboratory analysis. CEC analysis of loose material samples is commonly calculated via laboratory soil tests to understand the base cations and acid cations.

With all these methods, there is no one consistent approach to calculate the CEC. The existing approaches all have an extended degree of sample preparation in the laboratory and importantly, all these methods need to destroy the original sample material for calculating the CEC. Depending on the amount of fragmentation of the sample, these methods generate different results for the measured CEC.

Knowing the value of CEC for a given sample is important because the clays found either in the well or used as the mud are aluminosilicates in which some of the aluminum and silicon ions have been replaced by elements with different valence, or charge. For example, aluminum (Al⁺⁺⁺) may be replaced by iron (Fe⁺⁺) or magnesium (Mg⁺⁺), leading to a net negative charge. This charge attracts cations when the clay is immersed in an electrolyte, such as salty water, and causes an electrical double layer. The CEC is often expressed in terms of its contribution per unit pore volume, Qv. In formation evaluation, it is the contribution of cation-exchange sites to the formation electrical properties that is important.

Formation evaluation is performed to assess the quantity and producibility of fluids from a reservoir. Formation evaluation guides wellsite decisions, such as placement of perforations and hydraulic fracture stages, and reservoir development and production planning. In this regard, note that the cation-exchange process occurs in two stages during two phase flow in porous media. Initially, the charged sites of the internal surface of the clays establish a new equilibrium by exchanging cations with the aqueous phase. At later stages, the components of the aqueous and oleic phases compete for the charged sites on the external surface or edges of the clays. When there is sufficient time for the crude oil to interact with the rock (i.e., when the core is aged with the crude oil), a fraction of the charged sites are neutralized by the charged components stemming from the crude oil. Moreover, the positively charged calcite and dolomite surfaces (at the prevailing pH environment of the well) are covered with the negatively charged components of the crude oil and therefore less mineral dissolution takes place when oil is present in porous media. Thus, knowing the accurate value of the CEC for a given sample from a well is highly desirable as it allows the operator of the well to control the chemical composition of the mud for more efficiently detaching the oil from the underground rocks.

Therefore, there is a need for a new method that consistently determines the CEC value for a given sample, without destroying the sample. The method and associated system need to be simple to implement, inexpensive and reliable.

### BRIEF SUMMARY OF THE INVENTION

Accord to another embodiment, there is a method for calculating cation exchange capacity, CEC, associated with a mineral sample. The method includes receiving a material sample, altering the material sample to become a mineral sample that fits into an automated mineralogy and petrography system, analyzing the mineral sample with the automated mineralogy and petrography system to generate a mineral map, selecting only clays from the mineral map, calculating a total surface area of the clays in the mineral map, and calculating the CEC value of the material sample based on the total surface area of the clays of the mineral map and for all minerals in the mineral map.

According to another embodiment, there is an automated mineralogy and petrography system configured to calculate cation exchange capacity, CEC, associated with a mineral sample. The system includes an imaging system configured to receive a mineral sample, which is obtained by altering a material sample, and a computing system configured to receive measurements from the imaging system, analyze the mineral sample to generate a mineral map, select only clays from the mineral map, calculate a total surface area of the clays in the mineral map, and calculate the CEC value of the material sample based on the total surface area of the clays of the mineral map and for all minerals in the mineral map.

According to yet another embodiment, there is a non-transitory computer readable medium including computer executable instructions, wherein the instructions, when executed by a processor, implement the method noted above.

### BRIEF DESCRIPTION OF THE DRAWINGS

For a more complete understanding of the present invention, reference is now made to the following descriptions taken in conjunction with the accompanying drawings, in which:

FIG. 1 is a schematic diagram of an automated mineralogy and petrography system for rock analysis;

FIG. 2 illustrates a mineral map generated by the automated mineralogy and petrography system for a material sample;

FIG. 3 illustrates the mineral map from FIG. 2 in which all the minerals except the clays have been ignored and a surface area of each grain of clay is calculated;

FIG. 4 illustrates various types of clay and associated CEC values;

FIG. 5 illustrates a relationship between the surface area of the clay and the corresponding CEC value;

FIG. 6 illustrates comparative values between a traditional CEC estimation method and a novel method that uses the automated mineralogy and petrography system;

FIGS. 7A to 7C illustrate the same comparison between the traditional CEC estimation method and the novel method that uses the automated mineralogy and petrography system, but for individual well samples;

FIG. 8 show in a table the comparative results of the two methods for seven different wells;

FIG. 9 is a flow chart of a method for calculating the CEC value with the automated mineralogy and petrography system; and

FIG. 10 is a schematic diagram of a computing system that is part of the automated mineralogy and petrography system.

### DETAILED DESCRIPTION OF THE INVENTION

The following description of the embodiments refers to the accompanying drawings. The same reference numbers in different drawings identify the same or similar elements. The following detailed description does not limit the invention. Instead, the scope of the invention is defined by the appended claims. The following embodiments are discussed, for simplicity, with regard to a QEMSCAN™ (quantitative evaluation of minerals by scanning electron microscopy,) system that is capable to analyze a well sample, and is adapted to automatically calculate the CEC of the sample without fragmenting the sample. However, the embodiments to be discussed next are not limited to the QEMSCAN™ system, but may be applied to other automated mineralogy and petrography systems, like the RoqSCAN™ system, which is a mobile system.

Reference throughout the specification to “one embodiment” or “an embodiment” means that a particular feature, structure or characteristic described in connection with an embodiment is included in at least one embodiment of the subject matter disclosed. Thus, the appearance of the phrases “in one embodiment” or “in an embodiment” in various places throughout the specification is not necessarily referring to the same embodiment. Further, the particular features, structures or characteristics may be combined in any suitable manner in one or more embodiments.

According to an embodiment, an automated mineralogy and petrography system (e.g., QEMSCAN™) is configured to analyze the chemical and density data of a 2D material sample and determine specific minerals making up the sample, and also to record the size and X-Y position of the different mineral grains/particles on the 2D material sample's surface, to a micron-scale of precision. The system is also configured to measure the surface area of selected materials of the determined minerals (e.g., only the clays) and to calculate the CEC based on the measured surface area and a specific relation (to be discussed later). In this way, the CEC of the material sample is being calculated without destroying or fragmenting the sample, and the results are consistent as they do not depend on the fragmentation size of the sample. More details about the used system and the applied method are now presented.

The automated mineralogy and petrography system may include an imagining system, for example, an automated Scanning Electron Microscopy (SEM) mineralogy system. Such automated mineralogy and petrography system may be, for example, either the lab-based QEMSCAN™ machine or the moveable, wellsite RoqSCAN™ machine. Other known machines may be configured to achieve the CEC value based on the method to be discussed. No matter which machine is used, they are all configured to employ a non-destructive method, which preserves the rock material as a thin section or block, for future analysis. In other words, after performing the analysis of a certain material sample and its CEC is determined, the integrity of the full, entire material sample is preserved for later analysis.

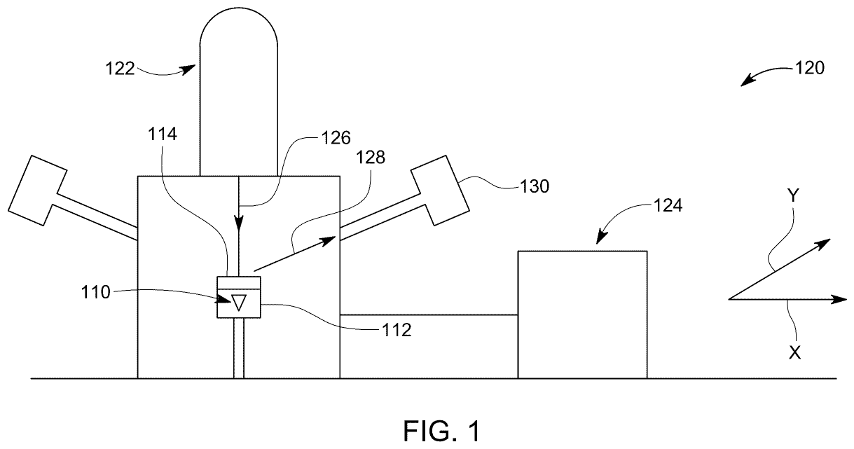

According to an embodiment, a rock and/or sediment material sample (e.g., from core, drill cuttings, etc.) 110, is being mounted in epoxy resin blocks or polished as glass thin sections 112, as illustrated in FIG. 1. The polished blocks 112 (called “sample” or “mineral sample” herein) may be ground down and further polished, for example, using a Sturers grinding/polishing machine. This step produces a flat, even surface which enables accurate electron dispersive scanning electron spectroscopy analysis (EDS-SEM). For the thin section, a piece of rock is first cut using a diamond saw and then attached with epoxy resin glue to a glass slide. The rock is then ground down and polished in the same way as above.

Next, the polished blocks and/or thin sections 112 are coated with a layer of carbon or other materials 114. Then, the coated probe 112 is placed inside the automated mineralogy and petrography systems system 120 and imaged using an imaging system (e.g., scanning electron microscope) 122. The analysis of the acquired information takes place in a computing system 124 that is connected to the imaging system 122. The computing system receives all the imaging information about the sample from the imagining system and stores mineralogic information about known materials.

An electron beam 126 (if the imaging system is a scanning electron microscope) fires electrons at each sample 112 and x-ray data 128 is produced, which is captured by detectors 130. The information from the detectors is transmitted to the computing system. The chemical and density data obtained for each sample is characteristic of specific minerals. A mineral dictionary (stored in the computing system) is then used to quantify the entire sample, i.e., each pixel of the sample is identified with a given mineral or chemical substance. The techniques for producing these results are machine specific and well described in the literature, and thus, their details are omitted herein.

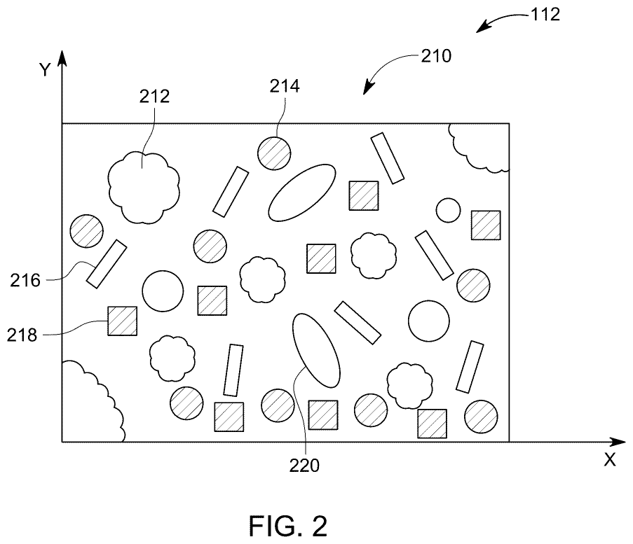

The analysis performed by the system 120 also records the size and the X-Y position of the various minerals on the 2D sample 112, to a micron-scale of precision, as shown for example in mineral map 210 in FIG. 2. More specifically, mineral map 210 in FIG. 2 corresponds to the surface of the sample 112 and represents plural minerals, for example, quartz 212, diopside 214, barite 216, pyrite 218 and clay 220. In other words, a mineral map is a representation of the minerals existing in the actual material sample, which is generated by the automated mineralogy and petrography system 120 based on (i) measurements obtained with the scanning electron microscope (or similar imaging device) that identifies a density of each pixel of the sample, and (ii) a mineral dictionary that maps each density to a corresponding mineral or substance. A material sample may include any number of other materials. The system 120 records the exact position X-Y of each mineral and its mass for that position. Knowing the mass of a given material at a certain location, and knowing the type of material, the system 120 is programmed to read the corresponding density of that material (for example, the density of quartz can be read from a table or database stored in the processor 124, as this density is known to be 2.66 g/cm³), and then to calculate the volume of that material in that sample.

Thus, the system 120 can calculate the volume percentage associated with each material in the sample 112. By adding all these volumes together, the system 120 is also able to calculate the total volume of the sample 112.

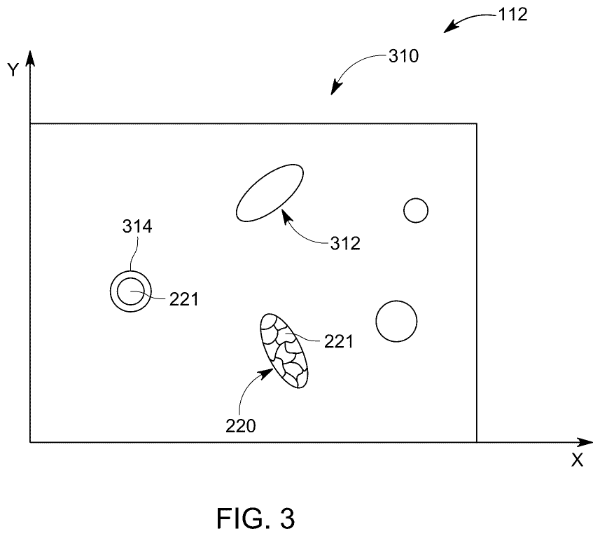

The system 120 can be also configured to provide the percentage amount (in mass or volume) of any material in the sample, for example, the clay 220 in the sample 112, and to create a model 310 of the sample 112 with all other materials or minerals removed, as shown in FIG. 3. Note that all minerals except the clay 220 have been removed in this model 310. In other words, the system 120 is programmed to manipulate the mineral map 210 and remove any desired material. Then, based on the selected clay map 310, the system 120 is configured to measure the total surface area 312 of all the clay present in the sample 112.

Because each pixel in the 2D SEM mineral/particle map 310 has mass and density data associated with it, as well as the XY area coordinates, the system is configured to calculate the 2D surface area of each clay grain/particle in mm⁻¹ (converted to equivalent m⁻¹). The total surface area 312 is calculated by estimating the individual surface area of each grain 221 of clay in the sample and then adding together the individual surface areas for all the grain clays. The individual surface area of a single grain of clay is estimated by drawing a line 314 around each clay particle/grain 221 and then measuring the 1D length of the line 314. Note that a clay 220 may include plural clay particles 221, as shown in FIG. 3. Thus, the line 314 is drawn around each clay particle 221, and not around a clay region 220. Where pixels of a particular clay mineral are connected together, they form the clay particle 221. If, for instance, a large circular-shaped illite particle/area had a small particle/area of smectite in its center, when calculating the size of the illite clay particle, the inner smectite particle would be ignored, i.e., the particle size would be the number of connected illite pixels. It is noted that the existing machines/algorithms are configured/set to separate joined particles, for instance, by a single pixel, or by considering the shape characteristics of the particle. The degree of strictness/leniency for these settings can be controlled by the operator of the machine. Note that the total surface area 312 in this embodiment gives the entire grain surface area, not just the area of the grains exposed on the sample's surface. Also note that the clay 220 includes different type of clay, each having a unique density and/or CEC value. The units and exponents then balance of the measured volume V (the volume of the entire clay in the sample 112) and the phase specific surface area (PSSA) of the clay, according to the formula: V×PSSA=total surface area (g/m³×m=g/m²), which provides a result in S.I. equivalent grams and meters. If there are various clay minerals in the considered sample, the automated mineralogy and petrography systems, for example, QEMSCAN, is flexible in terms of how the mineral data is quantified. In this instance, it is possible to call all of these clay minerals a single ‘mineral’ and process them together. For example, a typical QEMSCAN analysis identifies a couple of hundred mineral species. To make sense of these, the operator of the machine condenses them into smaller and smaller groups: e.g., smectite, illite, etc. The “smectite” comprises varies species of mineral that can be grouped together as smectite. Likewise, once the operator has quantified the final bulk clay minerals for a particular analysis (e.g., illite, smectite, kaolinite, chlorite, glauconite), it is possible to group all these groups together as a single “clay” grouping and perform quantitative measurements on this combined category. Thus, in one embodiment, all the various clay groups can be treated as a single clay having the cumulative volume V of all the groups. This volume is then used with the PSSA to calculate the total surface area.

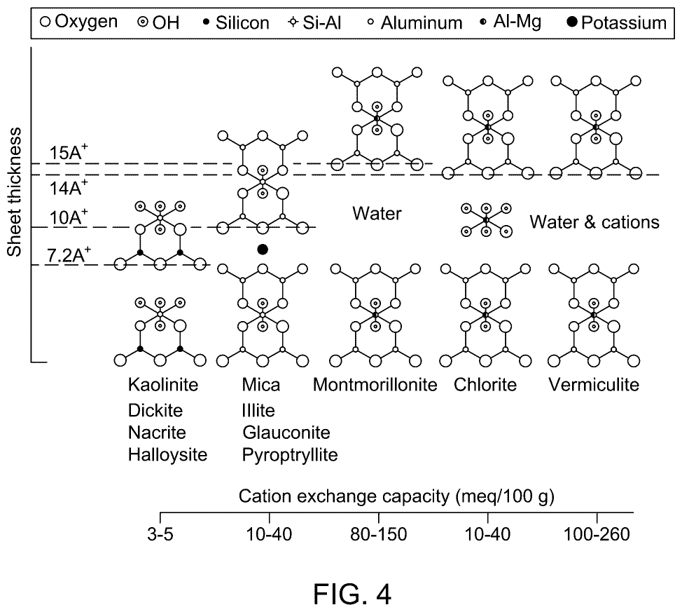

The PSSA is defined herein as the ratio of the individual surface area to the individual volume of a grain of clay in a given sample and has the units of mm 1 by convention. Note that each type of clay may have a different CEC, as illustrated in FIG. 4. Thus, the PSSA for each type of clay is different. In other words, for calculating the total surface area of the entire clay present in the given sample 112, it is not enough to know the clay regions, but also what type of clay is present at each pixel. Based on this data, the appropriate PSSA for each type of clay may be used to calculate the total surface area. For example, in one application, the total surface area is the sum of the individual surface areas of all the grains, where the individual surface area of a single grain is the product of the volume of that grain and the PSSA of that grain. The term “equivalent” is defined as the amount of substance that will react with Avogadro's number (NA) of hydrogen ions or electrons, respectively, in a given chemical reaction. Thus, the number of equivalents is unique for a given mass m and for a given substance (e.g., clay). As discussed above, for these calculations, only the clay data is used and all other mineralogic data is ignored as that data does not influence the CEC.

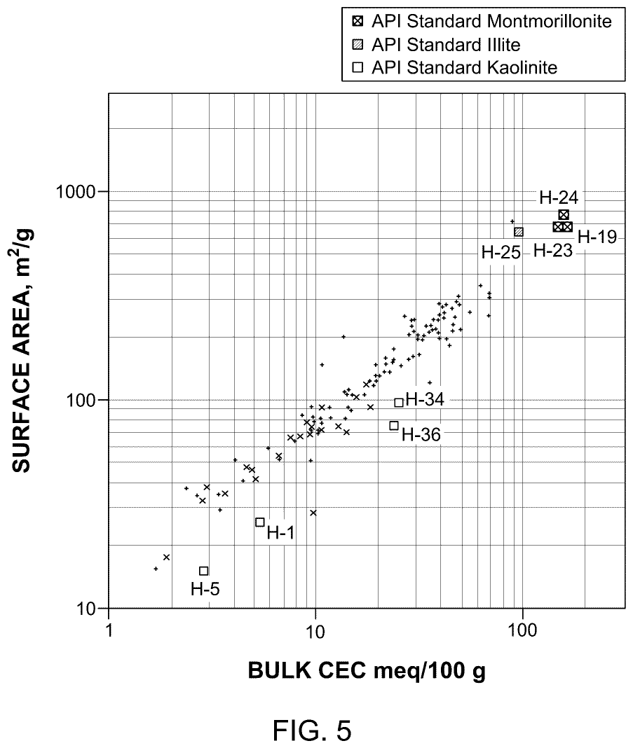

Next, the calculated total surface area for the clay 220 in the sample 112 is used by the system 120 to calculate the CEC. FIG. 5 shows that there is a relationship between the total surface area (plotted on the Y axis) and the CEC values (plotted on the X axis) and this relationship can be summarized as CEC=4.5 times the total surface area of the clay in a given sample having a total weight of 100 g. As the total surface area of the clay has been calculated above, the system 120 can be configured to calculate the CEC value by multiplying the calculated total surface area of the clay with 4.5. In this way, using any automated mineralogy and petrography system, it is possible to configure it to determine the clay areas, to calculate their surface area, and then to multiply the total surface area by 4.5 to determine the CEC for the considered sample.

The determination of CEC is important to the oil and gas systems for the reasons already discussed above, for example, well analysis, control and exploitation. In a first application, the electrical capacity of the clay effects the resistivity log response by reducing the resistivity of the rock. This can cause resistive hydrocarbons to be missed, which has large implications on reservoir net to gross (N: G) production and volumes. Note that drilling and extracting oil from a well is very capital intensive and thus, increasing the amount of oil that can be extracted is desirable.

Another application of the calculated CEC is in wellbore drilling. The clay swelling is a common problem encountered in the drilling process, often leading to hole collapse, lost time and significant remedial costs. Knowing the CEC value of the rock present in the well while drilling allows the resistivity effect to be compensated for, to potentially reveal bypassed hydrocarbons, and to predict the excess pressure or chemical remedial treatment to drilling mud required to counteract the clay swelling. This could be achieved, for example, by using at the wellsite the RoqSCAN™ system (which is a portable automated mineralogy and petrography system) and the above method, as RoqSCAN™ system is a ruggedized wellsite version of the lab-based QEMSCAN™ machine. This option also greatly reduces the analysis time because no samples need to be sent to a lab for standard XRD analysis, allowing for ‘real time’ wellsite decisions.

In yet another application, the CEC may be used for post-well petrophysical log analysis/correction. In this regard, away from the wellsite, CEC data combined with the routine automated mineralogy data delivery allows for much more accurate petrophysical log corrections, including dry and wet clay calculations and more accurate lithology descriptions (formation evaluation logs).

In still another application, it is possible to use the CEC for hydrogen and/or carbon storage and seal capacity estimation. The CEC data may be used when characterizing seal potential. Notably, the ability to characterize the impact of brine water and water geochemistry (salinity and pH) of pore water with CO₂ injection and storage. Another application of the CEC data is in the soil/peat testing, for environmental monitoring. The CEC analysis is also often using for testing important soil characteristics and fertility. Higher CEC values equate to a larger number of molecules (e.g., water, nutrients, herbicides) that are able to bind to the soil particle surface, and vice versa for lower CEC soils. As a result, farming approaches can be adapted to monitor and suit different soil characteristics, e.g., lower CEC soils need more and regular water irrigation compared to higher CEC soils as higher CEC soils can bind to more water and nutrients molecules. This has a direct impact on the volume and use of herbicide use and water irrigation. Both have consequently environmental impacts, e.g., direct use/cost of water and herbicides, and run-off into nearby water systems. CEC analysis can also be performed on peats. Detecting peat's physical and chemical ability on plant performance can be an important factor in preserving and re-forming peat lands, moving away from the use of peat in horticultural additive. Researching and applying CEC analysis using automated mineralogy and petrography systems, like QEMSCAN™ or RoqSCAN™, can also identify microplastic pollution in soils.

The automated mineralogy and petrography system-predicted CEC were checked against the ‘traditional’ cobalt hexamine trichloride (CHT) CEC. More specifically, a test to statistically compare the accuracy of how CEC is derived from the traditional CHT analysis versus CEC prediction from QEMSCAN™ analysis has been undertaken. For this purpose, core chips from in-house sections of conventional core from 7 exploration wells at the Norwegian North Sea basin were selected. Here, core samples from identical depths where split; one subset was sent for more traditional CEC measurement, by CHT analysis and the other subset, at the same depths in the same well, had QEMSCAN™ analysis performed on to predict the CEC value.

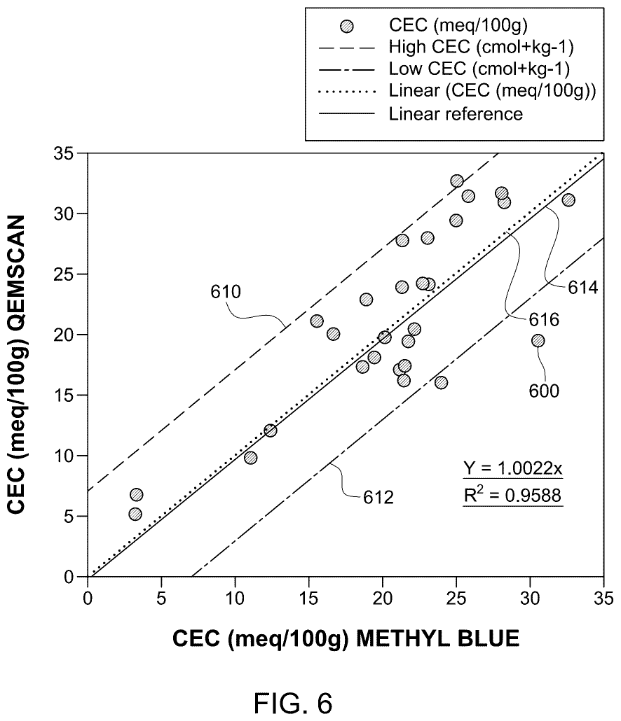

FIG. 6 shows points 600 corresponding to the QEMSCAN™ calculated CEC values (on the Y axis) versus the CHT calculated CEC values (on the X axis), and also the high CEC 610, the low CEC 612, a linear reference 614, and the linear CEC 616 (described by equation y=1.0022x). Overall, the results of this test are very promising with an R² of 0.9588 on 30 samples. Note that the combined CHT value was 20.64 while the QEMSCAN™-based value was 20.91. in one embodiment, the QEMSCAN™ CEC value has been calculated based on the difference of two terms A and B, where A is the calculated surface area for clays (as explained above) added to the calculated surface area for all minerals (including clays), add the result was divided by 2 (mean average between both). The term B is defined as the value of A divided by a constant, which was found to be 1.25. Thus, according to this embodiment, the step of multiplying the total surface area of the clay in a sample with 4.5 for calculating the CEC of the sample may be replaced by the step of calculating quantities A and B defined above and subtracting B from A to obtain the CEC.

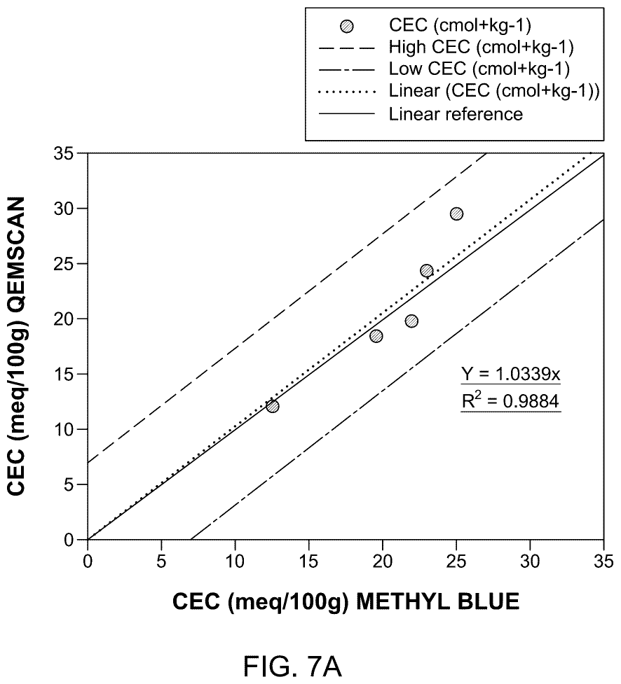

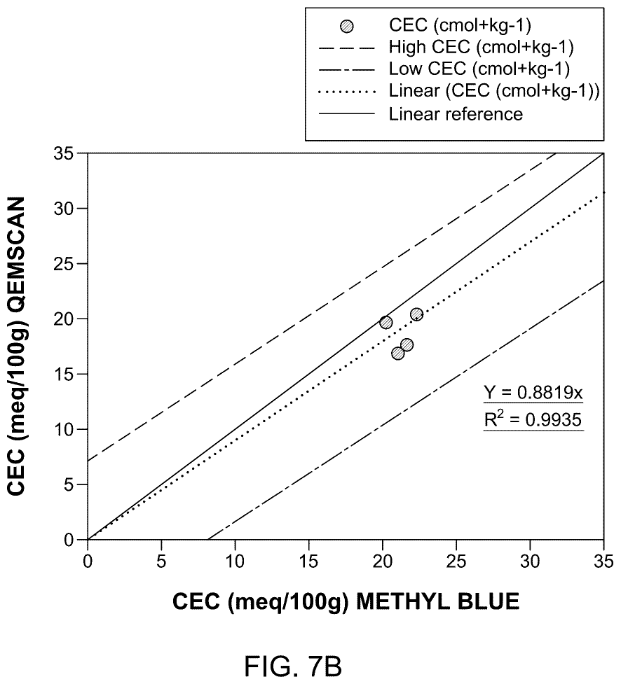

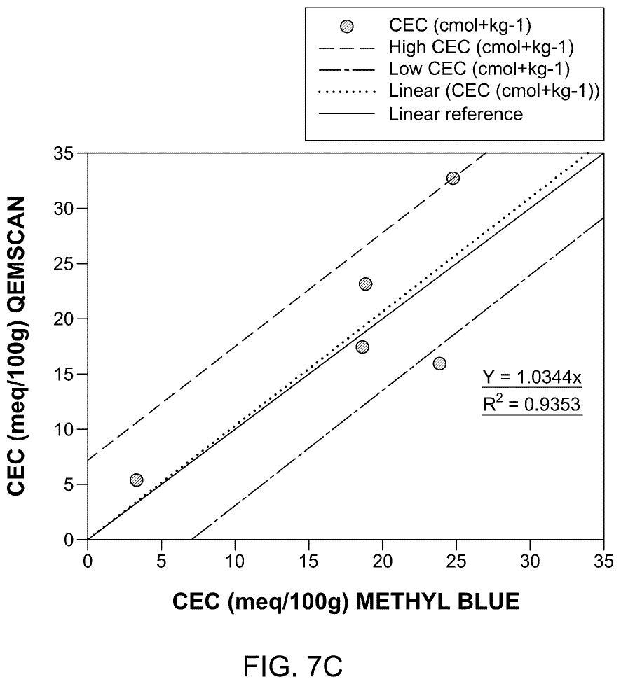

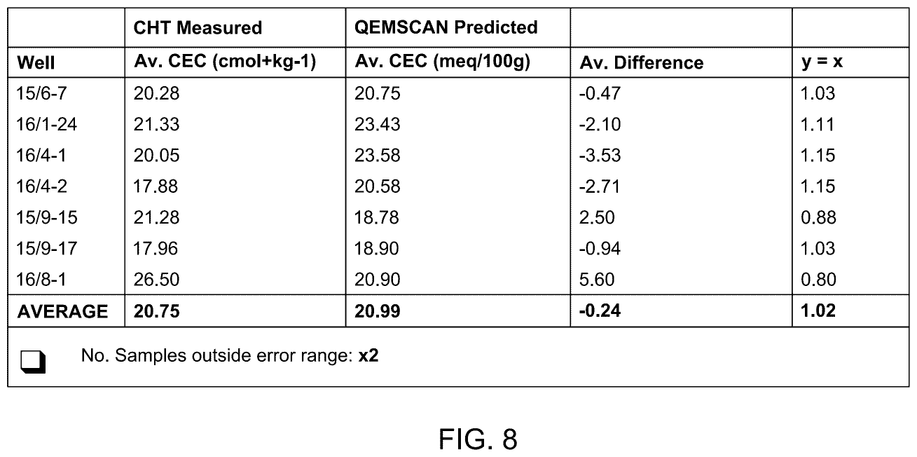

Individual well R² results are also promising, varying between 0.9936 and 0.9353, as shown in FIGS. 7A to 7C. Each figure shows the data for a different well, with the same quantities plotted as in FIG. 6. FIG. 8 summarizes the CHT measured CEC and the QEMSCAN™ predicted CEC and the average difference between the two is minimal. These results indicate that the novel process discussed above, when implemented into an automated mineralogy and petrography system, is capable to accurately and consistently determine the CEC of any sample.

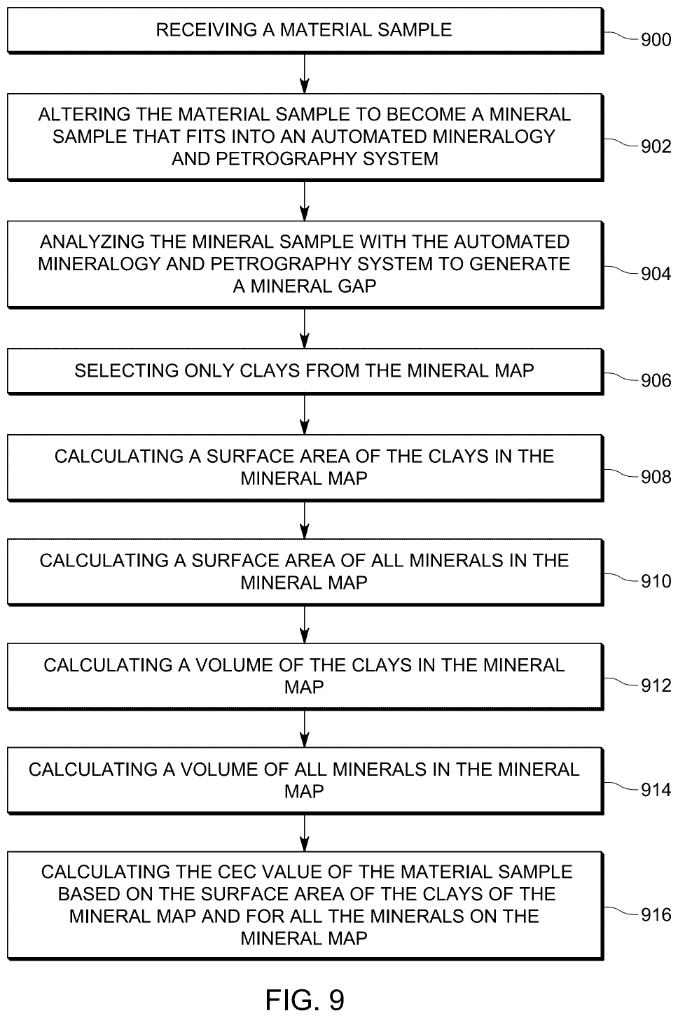

The CEC determination process discussed above is now summarized with regard to FIG. 9. The method includes a step 900 of receiving a material sample 110, a step 902 of altering the material sample 110 to become a mineral sample 112 that fits into an automated mineralogy and petrography system 120, a step 904 of analyzing the mineral sample 112 with the automated mineralogy and petrography system 120 to generate a mineral map 210, a step 906 of selecting only clays 220 from the mineral map 210, a step 908 of calculating a total surface area 312 of the clays 220 in the mineral map 210, a step 910 of calculating a surface area of all minerals in the mineral map, a step 912 of calculating a volume V of the clays of the mineral sample 112, a step 914 of calculating a volume of all minerals in the mineral map, and a step 916 of calculating the CEC value of the material sample 110 based on the total surface area of the clays 220 of the mineral map 210 and for all the minerals in the mineral map. One or more of the steps discussed may be omitted, for example, step 900 and/or step 902 and/or step 910.

In one application, the mineral sample is intact after the step of calculating the CEC value. The material sample is obtained from an oil well and the automated mineralogy and petrography system includes a scanning electron microscope. The mineral map represents plural minerals and clays, each pixel of the mineral map being associated with (1) a mineral or a clay, (2) a mass of the mineral or the clay, (3) a size of the mineral or the clay, and, only for the clay, with (4) a species of the clay. In one application, the mineral map includes plural clay species.

The step of calculating the total surface area includes drawing a line around each particle/grain of the clay, measuring a length of the line, assigning the measured length of the line to the PSSA of the grain, multiplying the volume of the grain with the corresponding PSSA to obtain the individual surface area of the grain, and adding together all the individual surface areas to obtain the total surface area of the clay. These steps are performed by the automated mineralogy and petrography system. The volume of the clays of the mineral sample is calculated by summing up individual volumes of each clay grain/particle in the sample, where an individual volume is calculated by dividing a mass of the clay gray/particle of the mineral sample with a density of the clay grain/particle of the mineral sample, as each type of clay may have different and/or unique densities and/or masses. The step of calculating the CEC value includes multiplying the total surface area of the clays with a constant, or calculating the quantities A and B discussed above and taking the difference A-B. The step of calculating the total surface area of the clays includes calculating a volume V of each clay grain of the mineral sample, multiplying the volume V with a phase specific surface area for each clay grain, and summing up the results of the multiplication to obtain the total surface area. The phase specific surface area is defined as a ratio of a local surface area a clay grain and a volume of the corresponding clay grain.

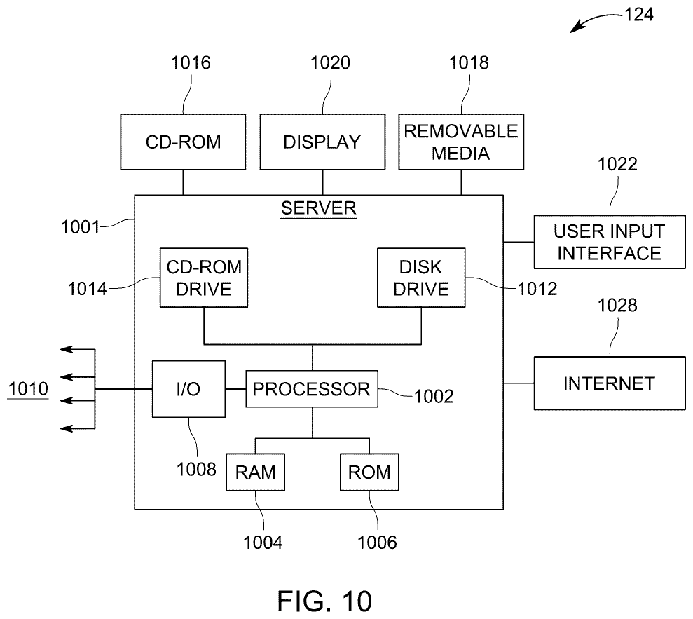

The methods discussed above for calculating the CEC of a given material sample may be implemented in the computing device 124, which is schematically illustrated in FIG. 10. Hardware, firmware, software or a combination thereof may be used to perform the various steps and operations described herein. Computing system 124 of FIG. 10 is an exemplary computing structure that may be used in connection with such a system.

Exemplary computing system 124 suitable for performing the activities described in the exemplary embodiments may include a server 1001. Such a server 1001 may include a central processor (CPU) 1002 coupled to a random access memory (RAM) 1004 and to a read-only memory (ROM) 1006. ROM 1006 may also be other types of storage media to store programs, such as programmable ROM (PROM), erasable PROM (EPROM), etc. Processor 1002 may communicate with other internal and external components through input/output (I/O) circuitry 1008 and bussing 1010 to provide control signals and the like. Processor 1002 carries out a variety of functions as are known in the art, as dictated by software and/or firmware instructions.

Server 1001 may also include one or more data storage devices, including hard drives 1012, CD-ROM drives 1014 and other hardware capable of reading and/or storing information, such as DVD, etc. In one embodiment, software for carrying out the above-discussed steps may be stored and distributed on a CD-ROM or DVD 1016, a USB storage device 1018 or other form of media capable of portably storing information. These storage media may be inserted into, and read by, devices such as CD-ROM drive 1014, disk drive 1012, etc. Server 1001 may be coupled to a display 1020, which may be any type of known display or presentation screen, such as LCD, plasma display, cathode ray tube (CRT), etc. A user input interface 1022 is provided, including one or more user interface mechanisms such as a mouse, keyboard, microphone, touchpad, touch screen, voice-recognition system, etc.

Server 1001 may be coupled to other devices, such as seismic sources, detectors, other imagining systems, etc. The server may be part of a larger network configuration as in a global area network (GAN) such as the Internet 1028, which allows ultimate connection to various landline and/or mobile computing devices.

The disclosed embodiments provide a novel method for calculating the CEC of a material sample without sample destruction or fragmentation. It should be understood that this description is not intended to limit the invention. On the contrary, the embodiments are intended to cover alternatives, modifications and equivalents, which are included in the spirit and scope of the invention as defined by the appended claims. Further, in the detailed description of the embodiments, numerous specific details are set forth in order to provide a comprehensive understanding of the claimed invention. However, one skilled in the art would understand that various embodiments may be practiced without such specific details.

Although the features and elements of the present embodiments are described in the embodiments in particular combinations, each feature or element can be used alone without the other features and elements of the embodiments or in various combinations with or without other features and elements disclosed herein.

This written description uses examples of the subject matter disclosed to enable any person skilled in the art to practice the same, including making and using any devices or systems and performing any incorporated methods. The patentable scope of the subject matter is defined by the claims, and may include other examples that occur to those skilled in the art. Such other examples are intended to be within the scope of the claims.

1. A method for calculating cation exchange capacity, CEC, associated with a mineral sample, the method comprising:
receiving a material sample;
altering the material sample to become a mineral sample that fits into an automated mineralogy and petrography system;
analyzing the mineral sample with the automated mineralogy and petrography system to generate a mineral map;
selecting only clays from the mineral map;
calculating a total surface area of the clays in the mineral map; and
calculating the CEC value of the material sample based on the total surface area of the clays of the mineral map and for all minerals in the mineral map.

2. The method of claim 1, wherein the mineral sample is intact after the step of calculating the CEC value.

3. The method of claim 1, wherein the material sample is from an oil well and the automated mineralogy and petrography system includes a scanning electron microscope.

4. The method of claim 1, wherein the mineral map represents plural minerals and clays, each pixel of the mineral map being associated with (1) a mineral or a clay, (2) a mass of the mineral or the clay, (3) a size of the mineral or the clay, and, only for the clay, with (4) a species of the clay.

5. The method of claim 4, wherein the mineral map includes plural clay species.

6. The method of claim 1, wherein the step of calculating the total surface area comprises:
drawing a line around each grain of the clay;
measuring a length of the line; and
assigning the length of the line as a value of a local surface area of the grain of the clay.

7. The method of claim 6, further comprising:
calculating a local surface area of each grain the clays of the mineral sample by multiplying an individual volume of each clay grain with the corresponding local surface area; and
adding all the local surface areas of the clays to obtain the total surface area,
wherein an individual volume of a clay grain is calculated by dividing a mass of the clay grain of the mineral sample to a density of the clay of the mineral sample, and
wherein each type of clay has a unique density.

8. The method of claim 1, wherein the step of calculating the CEC value includes multiplying the total surface area of the clays with a constant.

9. The method of claim 1, wherein the step of calculating the total surface area of the clays comprises:
calculating a volume V of each clay grain of the mineral sample;
multiplying the volume V with a phase specific surface area for each clay grain; and
summing up the results of the multiplication to obtain the total surface area,
wherein the phase specific surface area is defined as a ratio of a local surface area of a clay grain and a volume of the corresponding clay grain.

10. An automated mineralogy and petrography system configured to calculate cation exchange capacity, CEC, associated with a mineral sample, the system comprising:
an imaging system configured to receive a mineral sample, which is obtained by altering a material sample; and
a computing system configured to,
receive measurements from the imaging system,
analyze the mineral sample to generate a mineral map,
select only clays from the mineral map,
calculate a total surface area of the clays in the mineral map, and
calculate the CEC value of the material sample based on the total surface area of the clays of the mineral map and for all minerals in the mineral map.

11. The system of claim 10, wherein the mineral sample is intact after the step of calculating the CEC value.

12. The system of claim 10, wherein the material sample is from an oil well and the imaging system includes a scanning electron microscope.

13. The system of claim 10, wherein the mineral map represents plural minerals and clays, each pixel of the mineral map being associated with (1) a mineral or a clay, (2) a mass of the mineral or the clay, (3) a size of the mineral or the clay, and, only for the clay, with (4) a species of the clay.

14. The system of claim 13, wherein the mineral map includes plural clay species.

15. The system of claim 10, wherein the computing system is further configured to:
draw a line around each grain of the clay;
measure a length of the line; and
assign the length of the line as a value of local surface area of the grain of the clay.

16. The system of claim 15, wherein the computing system is further configured to:
calculate a local surface area of each clay grain of the mineral sample by multiplying an individual volume of each clay grain with the corresponding local surface area; and
adding all the local surface areas of the clays to obtain the total surface area,
wherein an individual volume of a clay grain is calculated by dividing a mass of the clay grain of the mineral sample with a density of the clay of the mineral sample, and
wherein each type of clay has a unique density.

17. The system of claim 10, wherein the computing system is further configured to multiply the total surface area of the clays with a constant to calculate the CEC value.

18. The system of claim 10, wherein the computing system is further configured to calculate the total surface area of the clays by:
calculating a volume V of each clay grain of the mineral sample;
multiplying the volume V with a phase specific surface area for each clay grain; and
summing up the results of the multiplication to obtain the total surface area,
wherein the phase specific surface area is defined as a ratio of a local surface area of a clay grain and a volume of the corresponding clay grain.

19. A non-transitory computer readable medium including computer executable instructions, wherein the instructions, when executed by a processor, implement a method for calculating cation exchange capacity, CEC, associated with a mineral sample, the medium comprising instructions for:
receiving a mineral sample that fits into an automated mineralogy and petrography system;
analyzing the mineral sample with the automated mineralogy and petrography system to generate a mineral map;
selecting only clays from the mineral map;
calculating a total surface area of the clays in the mineral map; and
calculating the CEC value of the material sample based on the total surface area of the clays of the mineral map and for all the minerals in the mineral map.

20. The medium of claim 19, wherein the step of calculating the total surface area of the clays comprises:
calculating a volume V of each clay grain of the mineral sample;
multiplying the volume V with a phase specific surface area for each clay grain; and
summing up the results of the multiplication to obtain the total surface area, wherein the phase specific surface area is defined as a ratio of a local surface area of a clay grain and a volume of the corresponding clay grain.
